(19) United States
(12) Patent Application Publication
(10) Pub. No.: US 2018/0088084 A1
(43) Pub. Date: Mar. 29, 2018

(54) FOOD DONENESS MONITOR

(71) Applicant: INTERNATIONAL BUSINESS MACHINES CORPORATION, Armonk, NY (US)

(72) Inventors: Amos Cahan, Dobbs Ferry, NY (US); Theodore G. van Kessel, Millbrook, NY (US)

(73) Assignee: INTERNATIONAL BUSINESS MACHINES CORPORATION, Armonk, NY (US)

(21) Appl. No.: 15/278,964

(22) Filed: Sep. 28, 2016

(51) Int. Cl. G01N 29/04; G01N 29/44; G01N 29/46; G01N 33/02

(52) U.S. Cl. CPC G01N 29/043; G01N 29/4436; G01N 29/4472; G01N 2291/0255; G01N 29/4481; G01N 33/02; G01N 2291/023; G01N 29/46

(57) ABSTRACT

Measuring and/or monitoring food doneness using ultrasound. The present invention provides a method, system, computer program product and sensor for measuring and/or monitoring the degree of food doneness. The present invention provides means for non-invasively and continuously determining the degree of food doneness using ultrasound technology. In general, the present invention works by applying ultrasound signals to a food item, receiving the ultrasound signals emitted back from the food item, and analyzing the input and output signals to determine the degree of food doneness. The food doneness sensor can be a stand-alone device, embedded into a cooking tool, embedded into a cooking apparatus, and/or embedded into a food production assembly line. The present invention can be used personally/commercially. User feedback regarding the performance of the present invention can be provided to a cloud database and used to modify/adjust the measuring/monitoring process. Finally, multiple ultrasound sensors/transponders can be coupled together.

### BACKGROUND

The present invention relates to the food industry and cooking tools. More specifically, the present invention relates to a method, system, computer program product and sensor for measuring and/or monitoring food doneness.

Food (e.g., meat, fish, cake, etc.) is often cooked before consumption. Some foods must be fully cooked before they can be safely consumed, while others can be safely consumed after being partially cooked to various degrees of personal preference. For example, cooking meat to the desired degree of doneness is considered important in preserving the meat taste and texture, especially in the case of steak preparation.

Cooking can eliminate disease-causing or pathogenic microorganisms. For example, chicken and pork meat must be thoroughly cooked before it can be safely consumed. Further, ground meat of any kind is particularly prone to harbor pathogenic bacteria after incomplete cooking because bacteria found on the surface, such as causative agents of infection (e.g. Salmonella species, E. coli, etc.), are dispersed throughout the meat after it has been ground.

It is often hard to accurately determine the degree of food doneness simply by inspecting the appearance of the food from the outside. Also, visually inspecting the interior part of the food requires cutting through it, which only provides information about the portion cut and may adversely affect the appearance and/or texture of the food.

Thermometers that are inserted into food can be used to measure the temperature in non-visible portions of the food. The temperature read from the thermometer can be used to estimate the degree of food doneness. However, thermometers are limited because they cannot be used to determine the degree of food doneness by measuring the temperature of the outside portion of the food and the thermometer must be inserted into the food.

Accordingly, there is a need for means to noninvasively and continuously determine the degree of food doneness, both for personal and commercial use (e.g. restaurants, food processing and manufacturing facilities, etc.).

### SUMMARY

The present invention relates to the use of ultrasound technology to measure and/or monitor the degree of food doneness before, during, and/or after the food has been cooked.

Accordingly, one aspect of the presenting invention is a method for determining food doneness using ultrasound, the method including: applying an input signal to a piezo element thereby causing the piezo element to generate a first ultrasound signal; applying, by the piezo element, the first ultrasound signal to a food element being cooked, in which the food element generates a second ultrasound signal responsive to receiving the first ultrasound signal; receiving, by the piezo element, the second ultrasound signal generated by the food element; and determining, by a computer, the degree of food doneness based on the first ultrasound signal and the second ultrasound signal.

The method can further include: determining whether the food element has reached the preferable or desired degree of food doneness.

The method can further include: obtaining user feedback, where the user feedback is used to adjust the cooking process of the food element and/or modify a calibration constant used by the computer; providing the user feedback to a cloud database; and updating the food doneness sensor based on the user's and/or other users' feedback.

Another aspect of the present invention is a computer program product for determining food doneness using ultrasound, the computer program product including a computer readable storage medium having program instructions embodied therewith, where the computer readable storage medium is not a transitory signal per se, and the program instructions readable/executable by a computer device to perform the method described above.

Another aspect of the present invention is a system for determining food doneness using ultrasound, the system including: a food doneness sensor; and a food doneness determining module, where the food doneness determining module is configured to perform the method described above.

Another aspect of the present invention is a food doneness sensor for determining the degree of food doneness of a food element, the food doneness sensor including: a supporting structure, a power source; an amplifier; a piezo element; a differential amplifier; a digital computer; and a transmitter.

In some embodiments of the present invention, the food doneness sensor can be embedded into a cooking tool, a cooking surface, and/or an industrial cooking line. Further, within the cooking tool, the piezo element can be a distance from a component placed in contact with a food element.

In some embodiments of the present invention, the food doneness sensor can further include a user interface, where a user can provide and/or receive performance feedback. Such performance feedback can be used to adjust/modify the food doneness sensor or system.

An advantage of the present invention is that the present invention can noninvasively and continuously measure and/or monitor the degree of food doneness with or without being in direct contact with the non-visible parts of a food element or item. Another advantage of the present invention is that it can efficiently, accurately, and quickly determine the degree of food doneness of a food item or element (e.g., a hamburger patty).

Another advantage of the present invention is that the sensor used for measuring and/or monitoring the degree of doneness does not have to be in close proximity to where the food is being cooked. In some embodiments of the present invention, the sensor is protected from heat, humidity and food remnants (e.g. cooking oil, butter, etc.) by being positioned in one end of a rod, in which the other end of the rod is in contact with the food.

### BRIEF DESCRIPTION OF THE DRAWINGS

FIG. 1 is a block diagram illustrating the basic components of an ultrasonic transponder or food doneness sensor according to an embodiment of the present invention.

FIG. 2 illustrates a cooking tool with a food doneness sensor incorporated therein according to an embodiment of the present invention.

FIG. 3 illustrates an industrial food production line with a food doneness sensor incorporated therein according to an embodiment of the present invention.

FIG. 4 is a flowchart of a method for measuring and/or monitoring food doneness using ultrasound according to an embodiment of the present invention. The STEPS contained in the broken dashed-lines are optional according to some embodiments of the present invention.

FIG. 5 is a block diagram illustrating hardware components of an exemplary computing system/device/server according to an embodiment of the present invention.

FIG. 6 illustrates a cloud computing environment according to an embodiment of the present invention.

FIG. 7 illustrates abstraction model layers according to an embodiment of the present invention.

FIG. 8 is a graph illustrating variation of an ultrasonic transmission spectrum of a food element or item due to material changes from cooking.

The subject matter which is regarded as the present invention is particularly pointed out and distinctly claimed in the Claims at the conclusion of the specification. The foregoing and other objects, features, and advantages of the present invention are made apparent through the following detailed description taken in conjunction with the accompanying drawings.

### DETAILED DESCRIPTION

The present invention relates to the food industry and cooking tools. More specifically, the present invention provides a method, system, computer program product and sensor for measuring and/or monitoring food doneness. The present invention provides means for non-invasively and continuously determining the degree of food doneness for a food item or element (e.g., a steak, cake, potatoes, fish, eggs, etc.). The present invention can be used personally or commercially.

An advantage of the present invention is that the present invention can noninvasively and continuously determine the degree of food doneness with or without being in direct contact with the non-visible parts of a food element or item.

Another advantage of the present invention is that it can efficiently, accurately, and quickly determine the degree of food doneness of a food item or element (e.g., a hamburger patty). In settings in which food must be rapidly prepared, such as in a fast food restaurant, it is important to rapidly prepare food while at the same time ensuring that the food is sufficiently cooked to the appropriate temperature. Food that is not cooked to the appropriate temperature can harbor microorganisms that can cause sickness or disease to those who consume the food. Cooking food to the required temperature or degree of doneness, as can be specified by the FDA, can destroy the harmful microorganisms found in food items. In other words, the present invention provides an automated and non-invasive means to effectively measure and monitor the internal temperature of food being cooked.

Another advantage of the present invention is that the sensor used for measuring and/or monitoring the degree of doneness does not have to be in close proximity to where the food is being cooked. In some embodiments of the present invention, the sensor is protected from heat, humidity and food remnants (e.g. cooking oil, butter, etc.) by being positioned in one end of a rod, in which the other end of the rod is in contact with the food.

In general, the present invention works by analyzing ultrasound waves emitted from a food element or item to determine the degree of food doneness. More specifically, an ultrasound transponder or sensor is used to measure and/or monitor the degree of food doneness by means of ultrasound technology, such as using elastography measurement techniques. The ultrasound transponder can be a standalone device or it can be coupled with a cooking tool (e.g. a fork). The ultrasound transponder can also be very small (measuring less than 1 cm in diameter). Further, the ultrasound transponder can be placed in contact with the surface of the food or inserted into the food.

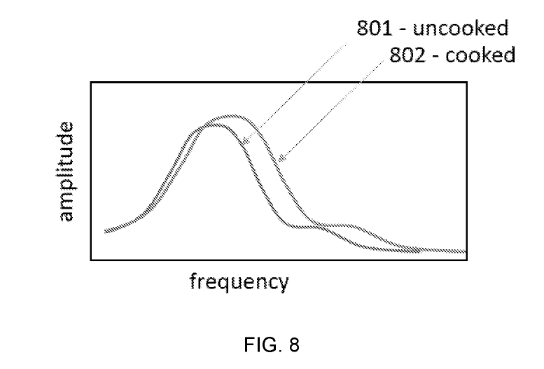

Cooking food causes proteins to congeal and this changes the mechanical and acoustic properties of the food. For example, meat becomes more rigid. In the simplest case, observing the losses of an ultrasonic transducer (or ultrasound transponder) in contact with cooked food (e.g., cooked meat) versus raw food (e.g., raw meat) over a range of frequencies or, alternatively, observing the transmission spectrum between two ultrasonic transducers will show very different acoustic spectra. In one embodiment of the present invention, this range of frequencies is varied from 50 kHz to 1 MHz. FIG. 8 illustrates this point.

Referring to FIG. 8, FIG. 8 is a graph illustrating variation of an ultrasonic transmission spectrum of a food element or item due to material changes from cooking. As the proteins in the cooked food become more rigid, it is common to see the ultrasonic transmission spectrum shift and change gradually in magnitude. As the food approaches doneness, the rate of change will slow down. These changes can be observed, calibrated and analyzed using methods familiar to one skilled in the art. This includes, but is not limited to, correlation methods, fourier methods and wavelet methods. Computer learning and analysis models can also be applied to improve the analysis including, but not limited to, neural networks, random forest classifiers, support vector and boosted support vector classifiers.

More elaborate measurements can be taken by superimposing a fast measurement signal on a much slower, and possibly higher amplitude, signal to allow the observation of how the acoustic properties change under pressure (e.g. Youngs Modulus). This second method is a variation on Elastography and provides additional information that can be used to discriminate between the various degrees of food doneness of cooked food (e.g., cooked meat) from raw food (e.g., raw meat).

The aforementioned measurements can be made simply by placing the ultrasonic transducer in contact with the food element or item or, alternatively, in transmission by utilizing 2 transducers (e.g., on opposing tines of a fork).

Elastography is a technique for measuring the firmness of an object, such as food. An ultrasound pulse, or other mechanical energy, is used to cause a distortion in the tissue of the object, after which the response of the tissue to the distortion is monitored by the ultrasound transponder and analyzed to infer the mechanical properties of the tissue. In the present invention, the response of the tissue to the ultrasound waves would be analyzed to determine the degree of food doneness.

In another embodiment of the present invention, the ultrasound transponder does not touch the food item at all (a non-contact method), but rather the ultrasonic signals are emitted from a distance (e.g. from the handle of a cooking tool such as a fork) and carried by a designated structure (e.g., a rod) to the food, in which reflected signals are carried back (in the opposite direction) to the ultrasound transponder. In this example, the fork is serving as a waveguide to transmit the acoustic energy from the ultrasound transponder to the food element, and then back to the ultrasound transponder. The ultrasound signals are then analyzed by the ultrasound transponder using computational means to determine the degree of food doneness. Note that in all cases it is preferred that either the ultrasound transponder itself, or an associated structure intended to conduct the acoustic energy, is in contact with the food element or item to be measured.

In another embodiment of the present invention, visual, sound and/or physical indicators can be used to inform the user about the degree of food doneness, the estimated time until the food will be done, recommend changes to the cooking energy being used (e.g. turn up or down the stovetop or oven temperature) and/or recommend changes to the food item itself (e.g., flip the food item and/or add an ingredient) in order to optimize the preparation of the food item being cooked. Examples of visual indicators include, but are not limited to, text displays, color cues, pictures of food items at various degrees of food doneness (e.g., displaying pictures of an inner portion of a steak cooked to rare, medium and well done degrees of doneness, in which these pictures show the degree of food doneness in real-time), etc. Examples of sounds indicators include, but are not limited to, alarms, bells, buzzers, voice indicators (e.g., a voice that announces the degree of food doneness reached or that provides directions to the user), songs or melodies, etc. An example of a physical indicator includes, but is not limited to, vibrations (e.g., causing an object to vibrate, such as a cell phone, to inform the user of the degree of food doneness).

In some embodiments of the present invention, the measuring and/or monitoring of the food item can be conducted continuously and/or intermittently at various time intervals.

Further, in some embodiments of the present invention, wired or wireless communication between the present invention (including the food doneness sensor) and other devices (e.g., a smartphone, computer, external device, etc.) can be supported, and information communicated to the other devices can be used in applications (e.g., cooking smart phone applications).

In another embodiment of the present invention, the present invention can be used to monitor the minimum, maximum and intermediate degrees of food doneness for a particular food item. For example, if the degree of food doneness has already reached a minimum threshold of doneness, the food can be monitored to ensure it is not cooked beyond a maximum threshold of doneness. For example, the present invention can indicate when a steak has reached the minimum appropriate degree of doneness for safe consumption (such as the steak being cooked to a rare degree of doneness), and then continue to monitor the steak and indicate when it has reached its maximum degree of doneness (such as the steak being cooked to a well-done degree of doneness). Once the steak has reached a well-done degree of doneness, the present invention can inform the user that the steak must be removed from the cooking element to prevent overcooking or burning the steak. Further, the present invention can indicate when the steak has reached intermediate degrees of food doneness (e.g., medium rare, medium, medium well, etc. degrees of doneness).

In some embodiments of the present invention, the type of food being cooked can be provided to the system by a user. Further, in some embodiments of the present invention, the present invention can be coupled with a camera (e.g. integrated with a smartphone) and an image processing system, in which the present invention can infer the type of food from an image and/or video. The present invention can be operatively coupled to a camera through wired and/or wireless communication means.

In another embodiment of the present invention, the present invention can be coupled to a cooking apparatus and/or surface and, through a feedback loop, the present invention can make adjustments to the cooking means (e.g., turn the temperature up or down of a stove-top or oven, or increase/decrease the pressure within a cooking apparatus such as a pressure cooker). The adjustment(s) to the cooking means can be done automatically. Further, the present invention can be operatively coupled to a cooking apparatus and/or surface through wired and/or wireless communication means.

In another embodiment of the present invention, the present invention can have a mechanical device attached and/or coupled to it, in which the mechanical device can remove food from the cooking area or turn off the cooking device(s) when the food has reached the desired degree of food doneness. The removing of the food from the cooking area or turning off of the cooking device(s) can be done automatically. The present invention can be operatively coupled to the mechanical means through wired and/or wireless communication means.

Some preferable embodiments of the present invention are described below in more detail with reference to the accompanying drawings, in which preferable embodiments of the present invention have been illustrated. However, the present invention can be implemented in various manners and, thus, cannot be construed to be limited to the embodiments disclosed herein. On the contrary, these embodiments of the present invention are provided for the thorough and complete understanding of the present invention and for completely conveying the scope of the present invention to those skilled in the art. The same reference generally refers to the same components in the exemplary embodiments of the present invention.

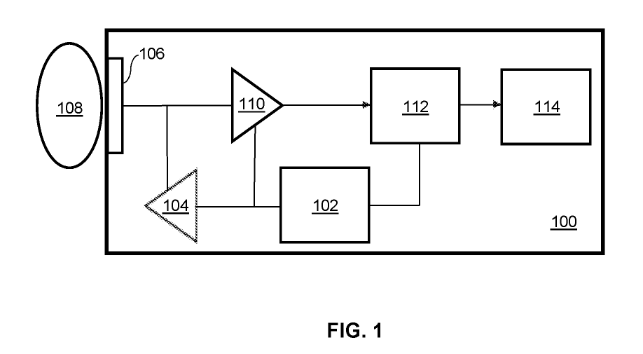

FIG. 1 illustrates the basic elements of the ultrasound transponder or food doneness sensor 100, hereinafter “sensor” 100. The sensor 100 at least includes a power source 102, an amplifier 104, a piezo element 106, a differential amplifier 110, a digital computer 112, and a transmitter 114. The sensor 100 is used to measure and/or monitor the degree of food doneness of food element (or item) 108. In cases where transmission measurement is desired, the circuit is replicated and operatively connected. Thus each channel can be synchronized to send and receive ultrasonic signals.

The basic elements of the sensor 100 are operatively coupled together. These basic elements can be coupled together through wired connections and/or wireless connections. Further these basic elements can be coupled together with intermediate components separating them. Those skilled in the art will understand various ways of coupling together the basic elements of the food doneness sensor which are considered contemplated by this disclosure. In one embodiment of the present invention the wireless coupling is accomplished with Bluetooth technology.

Note that there is a supporting structure which supports the piezo element and the circuit components. Further, the supporting structure is appropriately impedance matched to the device. In other words, the supporting structure provides mechanical support as well as has appropriate waveguide properties. Examples of supporting structures include, but are not limited to, a handle of a cooking fork, a handle of a cooking spatula, etc.

Referring to FIG. 1, an input frequency is applied via the power source 102 to an amplifier 104, which then applies the impedance matched input signal to piezo element 106 in physical contact with food element 108. Piezo element 106 vibrates in response to the voltage input from amplifier 104 thereby producing an ultrasonic acoustic signal. This ultrasonic acoustic signal traverses food element 108 and then also vibrates back the piezo element 106. This transmitted vibration causes a voltage to be produced in piezo element 106 (an output signal). The input signal is then compared to the output signal using digital computer 112 and differential amplifier 110. In other words, the output signal of piezo element 106 caused by the amplifier 104 is then compared to the input signal using digital computer 112 and differential amplifier 110, which is a measure of the acoustic transmission of the food element 108 at the input frequency. The input signal as compared to the output signal is then analyzed to determine the degree of food doneness. The transmitter 114 can then be used to inform the user or an external application/device regarding the degree of food doneness.

Food is a medium consisting of discrete or disunited vibrating particles known as scatterers. The scatterers vibrate upon the application of ultrasonic waves. The fundamental frequency of scatterers in the food medium changes with the change in the food's temperature. This change, or shift in fundamental frequency can be directly correlated with the change in food temperature. By observing or examining the change in fundamental frequency of a food item (e.g., steak or cake bread) while cooking and comparing it to food temperatures (possibly by using a thermometer), the present invention can be specifically calibrated/trained for certain food types. For example, a user or signal processing device/computer can determine what fundamental frequencies corresponds to a steak that has reached medium-rare doneness or a brownie that has reached the users preferred doneness (e.g., soft or crispy, etc.). Often, as illustrated in FIG. 8, a shift is observed accompanied by changes in magnitude. This signal trajectory is particularly useful when the aforementioned machine learning algorithms are used.

In some embodiments of the present invention, the food doneness sensor includes a database with the appropriate calibration constants for use with the various food types or items. Therefore, some embodiments of the food doneness sensor can include a user interface that allows the user to select the type of food(s) being cooked and enter any user preferences. Based upon the user's selections, the appropriate calibration constant will be obtained for use to determine the degree of food doneness of the particular food type.

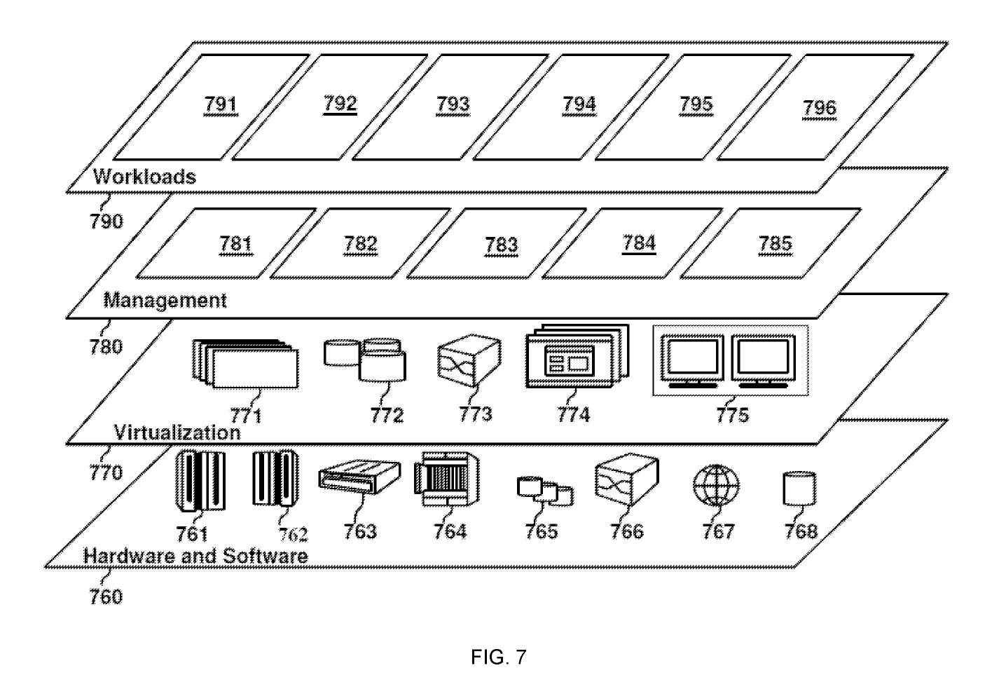

In some embodiments of the present invention, the food doneness sensor can be coupled to a user interface in which the user can provide feedback to the food doneness sensor system. This feedback can be used to train and/or calibrate the food doneness sensor system. The feedback can also be used to modify or adjust the cooking process for the food. For example, if a user requested that the food doneness sensor system beep only when a steak was cooked to “medium rare,” but the system beeped when the steak reached “medium,” the user could provide such feedback to adjust/modify the system. Further, in some embodiments of the present invention, the user feedback can be provided to a cloud database (see FIG. 7, food doneness sensor performance feedback processing layer 796) to help modify/adjust algorithms used in the food doneness sensor of other users' devices.

In some embodiments of the present invention, the food doneness sensor can be coupled with a camera (e.g. integrated with a smartphone) and an image processing system, in which the present invention can infer the type of food from an image and/or video. Further, in some embodiments of the present invention, the food doneness sensor can be coupled to a user interface in which the type of food being cooked can be provided by a user.

In another embodiment of the food doneness sensor, the piezo element 106 is not in direct physical contact with the food element 108. Rather, piezo element 106 can be positioned at some distance from the food, such as in the handle of a cooking tool. In this embodiment, the ultrasound wave generated by piezo element 106 is propagated through an ultrasound signal output channel to the distal part of the tool which is placed in contact with a food element 108 so as to protect piezo element 106 from high temperatures and/or humidity of food remnants that can be present in the immediate area where food element 108 is being cooked. In this case the channel acts as a waveguide. An exemplary embodiment of this includes placing the ultrasound transponder or sensor in the handle of a cooking fork and propagating the ultrasonic energy through the shaft to the tines of the fork. In another embodiment, the two transducer elements coupled to isolated shafts to operatively conduct ultrasound to a separate tine on the fork allows transmission measurements to be made through the food into which the fork is placed.

In another embodiment of the present invention, more than one sensor 100 are coupled together in order to measure and/or monitor the degree of food doneness of multiple food items at the same time (e.g., monitoring food in several different pots) and/or multiple different types of food items at the same time (e.g., a pot with different types of food items inside, such as steak, potatoes and carrots). Further, for example, when different portions of a cooking pot are exposed to different cooking energies (due to the distribution of heat generated by a flame underneath the pot), measuring and/or monitoring different portions of the pot in parallel can be helpful in determining the degree of doneness of food in each of the portions.

In some embodiments of the present invention the piezo element 106 is between about 0.05 to 5 cm in diameter, but preferably between about 1 to 2 cm in diameter. The size of the piezo element used can be varied to cover larger or smaller areas of the food element. For smaller food areas a 1 cm diameter is optionally used, and for larger food areas a 4 cm diameter is optionally used. By varying the diameter of the piezo element, this can ensure that that the innermost area of the food element is being measured to ensure thorough cooking.

In another embodiment of the present invention, the food doneness sensor is embedded or incorporated into a cooking apparatus (e.g., a stove, an oven, etc.).

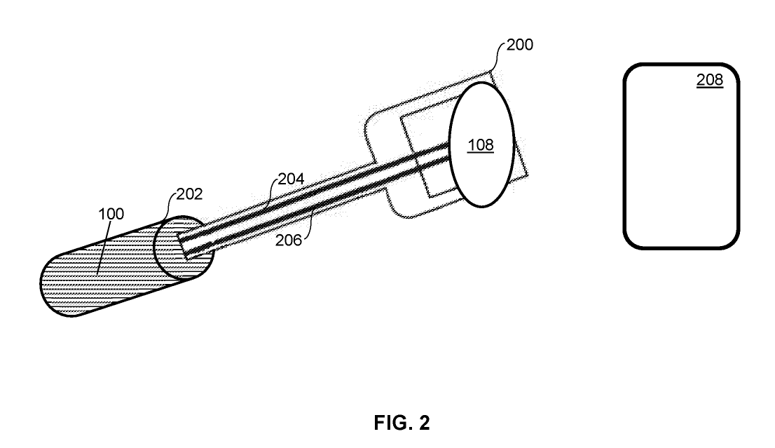

FIG. 2 illustrates the architecture of a cooking tool according to an embodiment of the present invention. More specifically, FIG. 2 illustrates a cooking tool with a food doneness sensor incorporated therein according to an embodiment of the present invention.

Referring to FIG. 2, sensor 100 is embedded within handle 202 (which can be a supporting structure) of cooking tool 200. The ultrasound wave generated by food doneness sensor 100 is propagated through ultrasound signal output channel (or acoustic output channel) 206 to the distal part of cooking tool 200, which is placed in contact with food element 108. By providing distance between handle 202 and food element 108, the food doneness sensor 100 is protected from high temperatures that can be present in the immediate area where food element 108 is being cooked. The acoustic signal generated by food element 108 when it is traversed by the output signal is propagated through ultrasound signal input channel (or acoustic input channel) 204 to the food doneness sensor 100 and is analyzed by food doneness sensor 100.

In another embodiment of the present invention, food doneness sensor 100 and/or cooking tool 200 is able to transmit information wirelessly, or through a wired connection, to an external device 208 (e.g., a smartphone), through which information can be presented to a user and/or further analyzed by the external device 208. In another embodiment, the user can also receive advice related to cooking the food to ensure that the food is cooked according to the user's preference. For example, if the user prefers that a steak has crispy edges, the external device could inform the user of changes to make to the cooking process to achieve crispy edges (e.g., turn up the temperature for a short period of time and/or baste the steak with olive oil).

In another embodiment of the present invention, food doneness sensor 100 is coupled to a cooking apparatus or surface and, through a feedback loop, the present invention can make adjustments to the cooking means (e.g., turn the temperature up or down of a stove-top and/or oven).

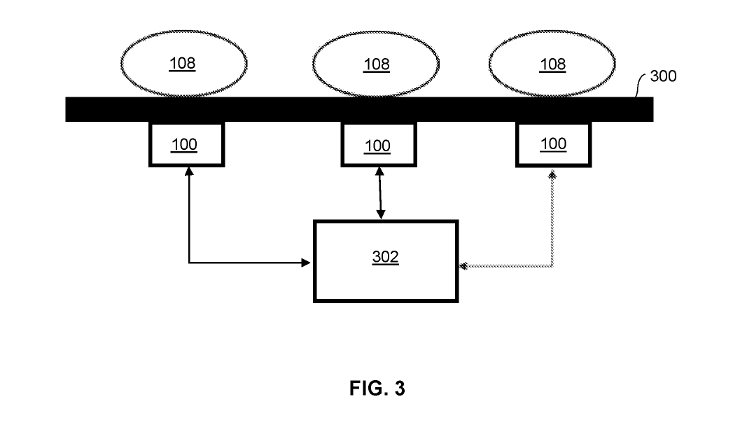

FIG. 3 illustrates the architecture of another embodiment of the present invention. More specifically, FIG. 3 illustrates an industrial food production line 300 with a food doneness sensor 100 incorporated therein according to an embodiment of the present invention.

Referring to FIG. 3, at least one food doneness sensor 100 is integrated into an industrial food production line 300, such that the food doneness sensor 100 can measure and/or monitor (either by direct contact or through acoustic signal propagation) the degree of food doneness of at least one food element 108. Food doneness sensor 100 transmits information to control unit 302. Control unit 302 processes the information collected from food doneness sensor 100. Control unit 302 is connected, either through wired or wireless connection, to industrial food production line 300.

In another embodiment of the present invention, control unit 302 can modify/adjust the cooking process to assure that the desired and/or required degree of doneness is reached.

In another embodiment of the present invention, industrial food production line 300 can have a mechanical device attached and/or coupled to it, in which the mechanical device can remove food from the cooking area or turn off the cooking device(s). The removing of the food from the cooking area or turning off of the cooking device(s) can be done automatically.

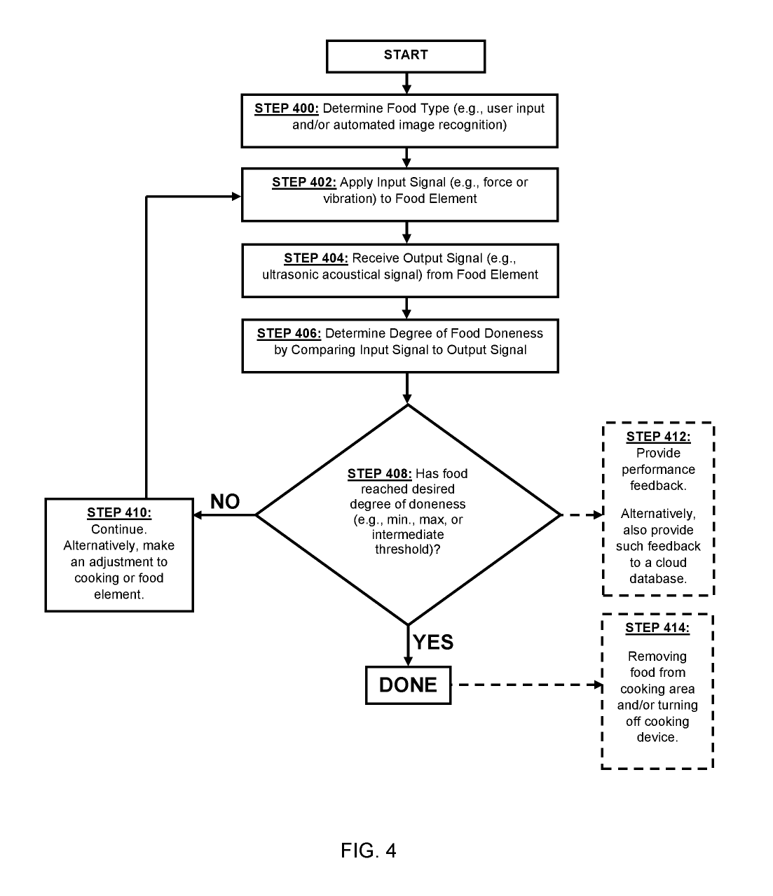

FIG. 4 is a flowchart of a method for measuring and/or monitoring food doneness using ultrasound according to an embodiment of the present invention. Such method can be a computer-implemented method.

Referring to FIG. 4, in STEP 400, the food type is determined. In some embodiments of the present invention, the food type is determined by image processing, in which the present invention can infer the type of food from an image and/or video. Further, in some embodiments of the present invention the food type is determined by user provided input. Further, in some embodiments of the present invention the food type is determined by analyzing the food item.

In STEP 402, an input signal is applied to a food element (e.g., meat, cake, etc.). More specifically, in STEP 402 an input frequency can applied to an amplifier, via a power source, which then applies an impedance matched input signal to a piezo element. The piezo element can be in physical contact with the food element. The piezo element vibrates in response to the voltage input from the amplifier thereby producing an ultrasonic acoustic signal. The ultrasonic signal then traverses the food element.

In STEP 404, an output signal is received from the food element. More specifically, in STEP 404 the ultrasonic signal that traverses the food element then vibrates back to the piezo element. The piezo element then produces an output signal. In other words the piezo element vibrating back causes another voltage signal in the piezo element. The differential amplifier then receives the output signal from the piezo element.

In STEP 406, the degree of food doneness is determined by analyzing the input signal as compared to the output signal. More specifically, in STEP 406, the differential amplifier and digital computer are used to determine the degree of food doneness. The digital computer analyzes the data obtained from the differential amplifier and determines the degree of food doneness. In some embodiments of the present invention, the degree of food doneness can be measured by using elastography measurement techniques.

In STEP 408, determining whether the food has reached the desired degree of doneness. More specifically, in STEP 408, based on the specific calibration/training for the food element or item, the system indicates whether the food has reached the desired degree of food doneness. If “yes,” an indication of yes is signaled to the user and/or cooking apparatus. If “no,” the method proceeds to STEP 410, in which STEPS 402-408 are repeated. The indication of “no” can also be signaled to the user and/or cooking apparatus.

Some embodiments of the present invention can include STEP 412. In STEP 412, providing performance feedback to the system. In some embodiments of the present invention, a user can provide the performance feedback to the system via a user interface. This feedback can be used to train and/or calibrate the food doneness sensor system. Further, in some embodiments of the present invention, the user feedback can be provided to a cloud database to help modify/adjust the food doneness measuring/monitoring system/method of other users' devices (see FIG. 7, food doneness sensor performance feedback processing layer 796).

Some embodiments of the present invention can include STEP 414. In STEP 414, removing food from the cooking area or turning off the cooking device(s). The removing of the food from the cooking area or turning off of the cooking device(s) can be done automatically.

In another embodiment of the present invention an indication of yes or no is signaled to the user via visual indicators (e.g., text words, light colors), sound indicators (e.g., alarm, voice, etc.), and/or physical indicators (e.g., vibration, etc.).

In another embodiment of the present invention, the present invention can indicate when a minimum, maximum or intermediate threshold doneness is reached. For example, if a restaurant serves steaks. The user can set the preferred doneness for the steaks to be cooked between rare to well done. The present invention could signal to the user that the steak has reached a minimum threshold of rare, and then signal to the user when the steak has reached a maximum threshold of well done and must be removed from the cooking apparatus before it is burned beyond possible use.

In another embodiment of the present invention, the present invention can indicate to adjust the food element (e.g., flip the food over, add sauce, etc.), adjust the cooking tool (e.g., change area of contact with food item, etc.), and/or adjust the cooking apparatus (e.g., lower the heat, increase the heat, etc.).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers.

A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

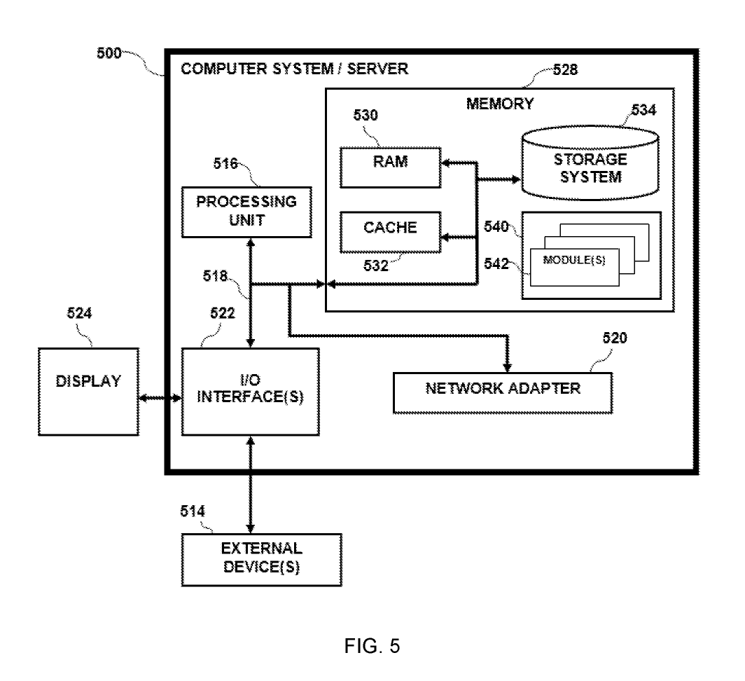

Referring to FIG. 5, FIG. 5 is a block diagram of an exemplary computer system/server 500 in detail, which is applicable to implement the embodiments of the present invention. Computer system/server 500 is only illustrative and is not intended to suggest any limitation as to the scope of use or functionality of embodiments of the invention described herein.

As shown in FIG. 5, computer system/server 500 is shown in the form of a general-purpose computing device. The components of computer system/server 500 can include, but are not limited to, one or more processors or processing units 516, a system memory 528, and a bus 518 that couples various system components including system memory 528 to processor 516.

Bus 518 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 500 typically includes a variety of computer system readable media. Such media can be any available media that is accessible by computer system/server 512 and it includes both volatile and non-volatile media, removable and non-removable media.

System memory 528 can include computer system readable media in the form of volatile memory, such as random access memory (RAM) 530 and/or cache memory 532. Computer system/server 512 can further include other removable/non-removable, volatile/non-volatile computer system storage media. By way of example only, storage system 534 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”) and an optical disk drive for reading from or writing to a removable, non-volatile optical disk such as a CD-ROM, DVD-ROM or other optical media can be provided. In such instances, each can be connected to bus 518 by one or more data media interfaces. As will be further depicted and described below, memory 528 can include at least one program product having a set (e.g., at least one) of program modules that are configured to carry out the functions of embodiments of the invention.

Program/utility 540, having a set (at least one) of program modules 542, can be stored in memory 528 by way of example, and not limitation, as well as an operating system, one or more application programs, other program modules, and program data. Each of the operating system, one or more application programs, other program modules, and program data or some combination thereof, can include an implementation of a networking environment. Program modules 542 generally carry out the functions and/or methodologies of embodiments of the invention as described herein.

Computer system/server 512 can also communicate with one or more external devices 514 (such as a keyboard, a pointing device, a display 524, etc.), one or more devices that enable a user to interact with computer system/server 500, and/or any devices (e.g., network card, modem, etc.) that enable computer system/server 500 to communicate with one or more other computing devices. Such communication can occur via Input/Output (I/O) interfaces 522. Still yet, computer system/server 500 can communicate with one or more networks such as a local area network (LAN), a general wide area network (WAN), and/or a public network (e.g., the Internet) via network adapter 520. As depicted, network adapter 520 communicates with the other components of computer system/server 500 via bus 518. It should be understood that although not shown, other hardware and/or software components could be used in conjunction with computer system/server 500. Examples, include, but are not limited to, microcode, device drivers, redundant processing units, external disk drive arrays, RAID systems, tape drives, and data archival storage systems, etc.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

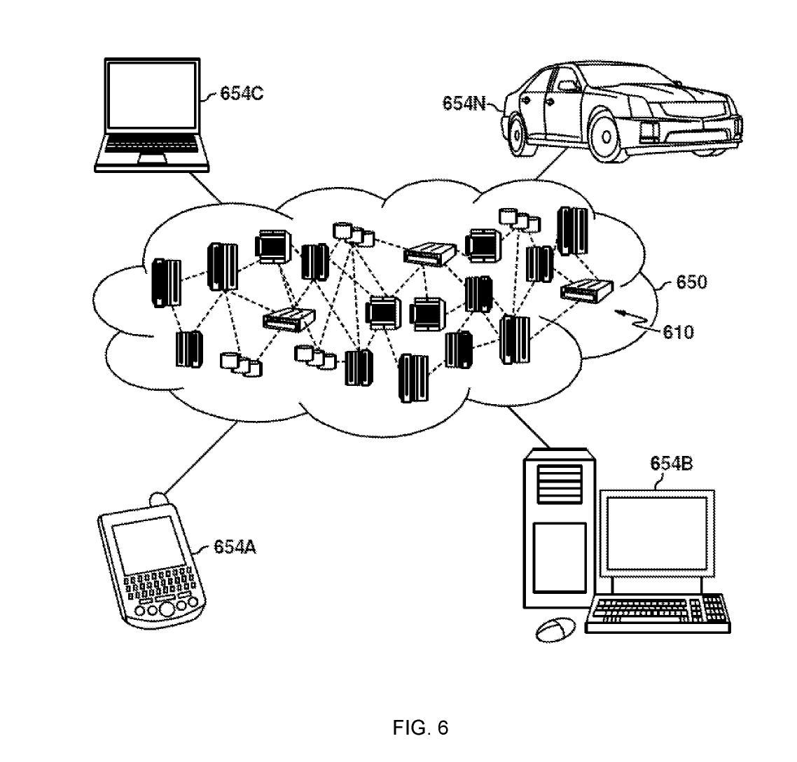

Referring now to FIG. 6, illustrative cloud computing environment 650 is depicted. As shown, cloud computing environment 650 includes one or more cloud computing nodes 610 with which local computing devices used by cloud consumers, such as, for example, personal digital assistant (PDA) or cellular telephone 654A, desktop computer 654B, laptop computer 654C, and/or automobile computer system 654N may communicate. Nodes 610 may communicate with one another. They may be grouped (not shown) physically or virtually, in one or more networks, such as Private, Community, Public, or Hybrid clouds as described hereinabove, or a combination thereof. This allows cloud computing environment 650 to offer infrastructure, platforms and/or software as services for which a cloud consumer does not need to maintain resources on a local computing device. It is understood that the types of computing devices 654A-N shown in FIG. 6 are intended to be illustrative only and that computing nodes 610 and cloud computing environment 650 can communicate with any type of computerized device over any type of network and/or network addressable connection (e.g., using a web browser).

Referring now to FIG. 7, a set of functional abstraction layers provided by cloud computing environment 650 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 760 includes hardware and software components. Examples of hardware components include: mainframes 761; RISC (Reduced Instruction Set Computer) architecture based servers 762; servers 763; blade servers 764; storage devices 765; and networks and networking components 766. In some embodiments, software components include network application server software 767 and database software 768.

Virtualization layer 770 provides an abstraction layer from which the following examples of virtual entities may be provided: virtual servers 771; virtual storage 772; virtual networks 773, including virtual private networks; virtual applications and operating systems 774; and virtual clients 775.

In one example, management layer 780 may provide the functions described below. Resource provisioning 781 provides dynamic procurement of computing resources and other resources that are utilized to perform tasks within the cloud computing environment. Metering and Pricing 782 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 783 provides access to the cloud computing environment for consumers and system administrators. Service level management 784 provides cloud computing resource allocation and management such that required service levels are met. Service Level Agreement (SLA) planning and fulfillment 785 provide pre-arrangement for, and procurement of, cloud computing resources for which a future requirement is anticipated in accordance with an SLA.

Workloads layer 790 provides examples of functionality for which the cloud computing environment may be utilized. Examples of workloads and functions which may be provided from this layer include: mapping and navigation 791; software development and lifecycle management 792; virtual classroom education delivery 793; data analytics processing 794; transaction processing 795; and food doneness sensor performance feedback processing layer 796.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A food doneness sensor for determining the degree of food doneness of a food element, the food doneness sensor comprising:
a supporting structure
a power source;
an amplifier;
a piezo element;
a differential amplifier;
a computer; and
a transmitter.

2. The food doneness sensor according to claim 1, wherein the food doneness sensor is embedded into a cooking tool.

3. The food doneness sensor according to claim 2, wherein the cooking tool comprises:
an acoustic channel, wherein the acoustic channel propagates ultrasound acoustic waves, and wherein the piezo element is located a distance from a component placed in contact with the food element.

4. The food doneness sensor according to claim 1, the food doneness sensor further comprising:
a user interface, wherein a user can provide and/or receive performance feedback information.

5. The food doneness sensor according to claim 1, wherein the food doneness sensor is embedded into a cooking system, wherein the cooking system is selected from the group consisting of a cooking apparatus, cooking surface and an industrial cooking line.

6. The food doneness sensor according to claim 5, wherein the cooking system comprises means for automatically adjusting the cooking temperature.

7. The food doneness sensor according to claim 5, wherein the cooking system comprises means for automatically adjusting the food element.

8. The food doneness sensor according to claim 1, wherein the piezo element is about 0.05 cm to 5 cm in diameter.

9. The food doneness sensor according to claim 8, wherein the piezo element is about 1 cm to 2 cm in diameter.

10. The food doneness sensor according to claim 1, wherein the food doneness sensor is coupled to one or more other food doneness sensors.

11. The food doneness sensor according to claim 1, wherein the amplifier is coupled to the piezo element, and the amplifier applies an input signal to the piezo element.

12. The food doneness sensor according to claim 11, wherein the piezo element is configured to apply a first ultrasound signal to the food element whereby the food element generates a second ultrasound signal responsive to the first ultrasound signal, and wherein the piezo element is configured to receive the second ultrasound signal generated by the food element.

13. The food doneness sensor according to claim 12, wherein the computer is configured to determine the degree of food doneness based on the first ultrasound signal and the second ultrasound signal.

14. The food doneness sensor according to claim 13, wherein the computer uses elastography measurement theory to compare the first ultrasound signal and the second ultrasound signal.

15. The food doneness sensor according to claim 13, wherein the computer uses analytic methods to evaluate the food doneness based on the observed ultrasonic signal that include at least one of a correlation analysis method, a fourier analysis method, and a wavelet analysis method.

16. The food doneness sensor according to claim 15, wherein the computer uses machine learning methods including at least one of a neural network, a random forest classifier, a support vector classifier, and a boosted support vector classifier.

17. The food doneness sensor according to claim 1, wherein the transmitter is coupled to an external application and/or device whereby the transmitter transmits information regarding the degree of food doneness.

18. The food doneness sensor according to claim 17, the food doneness sensor further comprising:
a user interface configured to allow a user to select a type of food element being cooked.

19. The food doneness sensor according to claim 1, the food doneness sensor further comprising:
a signaling means:
wherein the signaling means is selected from the group consisting of a visual indicator means, a sound indicator, and a physical indicator means;
wherein the signaling means activates a visual, sound and/or physical indicator signaling the degree of food doneness.

20. The food doneness sensor according to claim 19:
wherein the signaling means is configured to indicate when a threshold doneness has been met, and
wherein the threshold is selected from the group consisting of a minimum threshold, a maximum threshold, or an intermediate threshold.
